(19) United States
(12) Patent Application Publication
(10) Pub. No.: US 2023/0175914 A1
(43) Pub. Date: Jun. 8, 2023

(54) SYSTEM AND METHOD FOR GAS DETECTION AT A FIELD SITE USING MULTIPLE SENSORS

(71) Applicant: BP America Production Company, Houston, TX (US)

(72) Inventors: Justice Diven, Evergreen, CO (US); Scott Feldman-Peabody, Golden, CO (US); Zach Wilcock, Denver, CO (US); David Cox, Cypress, TX (US); Sarah O'Neil, Lynnfield, MA (US)

(21) Appl. No.: 17/540,348

(22) Filed: Dec. 2, 2021

(51) Int. Cl. G01M 3/24; G08B 13/16

(52) U.S. Cl. CPC G01M 3/24; G08B 13/1672

(57) ABSTRACT

Disclosed herein are systems and methods for detecting a gas leak at a field site, the system having at least one gas sensor, an acoustic sensor, and a processing unit having a processor and a non-transitory computer readable storage media storing instructions, the processing unit configured to receive signals from the at least one gas sensor and the acoustic sensor. When the instructions are executed by the processor of the processing unit, the signals received from the at least one gas sensor and the acoustic sensor are analyzed to determine a presence of a gas leak, the presence of the gas leak determined if at least one signal received from the gas sensor is above a first gas threshold value and at least one signal received from the acoustic sensor is above a first acoustic threshold value.

### BACKGROUND

Reduction of rogue methane emissions from oil and gas well sites is desirable to protect the environment. Leaks can happen in the field due to equipment failure or malfunction. When this happens, it is desirable to “shut-in” (i.e., turn off) the well in order to minimize the emission volume. Many of these well sites run autonomously in extremely remote areas with limited data connectivity and far from human intervention. It is desirable to be able to detect these emissions remotely, quickly, and autonomously, and if possible, shut in the well automatically. Because sending people on site is expensive, it is also important to reduce the number of false alarms from any methane detection system.

Therefore, a need exists for a system and method of gas detection having multiple sources and types of detection, the system and method designed to increase the likelihood of detection of a gas leak while reducing the number of false alarms. It is to such a system and method that the presently disclosed inventive concepts are directed.

### SUMMARY

A system for detecting a gas leak is disclosed, the system comprising: at least one gas sensor; an acoustic sensor; and a processing unit having a processor and a non-transitory computer readable storage media storing instructions, the processing unit configured to receive signals from the at least one gas sensor and the acoustic sensor; wherein the instructions, when executed, cause the processor of the processing unit to analyze signals received from the at least one gas sensor and the acoustic sensor to determine a presence of a gas leak, the presence of the gas leak determined if at least one signal received from the gas sensor is above a first gas threshold value and at least one signal received from the acoustic sensor is above a first acoustic threshold value.

The system further comprising a communication device and wherein the instructions further cause the processing unit to send an alarm to a predetermined recipient when the processing unit determines the presence of the gas leak.

The system, wherein the instructions further cause the processing unit to send a signal via the communication device to a valve of a well causing the valve to close when the processing unit determines the presence of the gas leak.

The system, wherein the instructions, when executed, further cause the processor of the processing unit to analyze the signals received from the at least one gas sensor and the acoustic sensor to determine the presence of a gas leak, the presence of the gas leak determined if at least one signal received from the gas sensor is above a second gas threshold value higher than the first gas threshold value, or at least one signal received from the acoustic sensor is above a second acoustic threshold value higher than the first acoustic threshold value.

A method of identifying a gas leak, comprising: receiving input from at least one gas sensor, the input indicative of a presence of an amount of gas in the air at a field site; analyzing the input from the at least one gas sensor to determine if the amount of gas in the air is above a first gas threshold value; receiving input from an acoustic sensor, the input indicative of a sound at the field site; analyzing the input from the acoustic sensor to determine if the sound is above a first acoustic threshold value; and sending an alarm to a predetermined recipient if it is determined that the gas is above the first gas threshold value and the sound is above the first acoustic threshold value, the alarm indicative of a presence of a gas leak at the field site.

The method further comprising: analyzing the input from the at least one gas sensor to determine if the amount of gas in the air is above a second gas threshold value higher than the first gas threshold value; analyzing the input from the acoustic sensor to determine if the sound is above a second acoustic threshold value higher than the first gas threshold; and sending the alarm to the predetermined recipient if it is determined that the gas is above the second gas threshold value or the sound is above the second acoustic threshold value, the alarm indicative of a presence of a gas leak at the field site.

### BRIEF DESCRIPTION OF THE SEVERAL VIEWS OF THE DRAWINGS

To assist those of ordinary skill in the relevant art in making and using the subject matter hereof, reference is made to the appended drawings, which are not intended to be drawn to scale, and in which like reference numerals are intended to refer to similar elements for consistency. For purposes of clarity, not every component may be labeled in every drawing.

FIG. 1 is a diagrammatic view of hardware forming an exemplary embodiment of a system for detecting gas leaks at a field site constructed in accordance with the present disclosure.

FIG. 2 is a diagrammatic view of an exemplary detection device for use in the system for detecting gas leaks at the field site illustrated in FIG. 1.

FIG. 3 is a diagrammatic view of an exemplary embodiment of a host system for use in the system for detecting gas leaks at the field site illustrated in FIG. 1.

FIG. 4 is a process diagram of an exemplary acoustic gas leak detection process where sound data is processed by a host system in accordance with one aspect of the present disclosure.

FIG. 5 is a process diagram of an exemplary acoustic gas leak detection process with automated shutoff of a well in accordance with one aspect of the present disclosure.

FIG. 6 is a process diagram of an exemplary gas leak detection process using data from an acoustic sensor and a gas sensor in accordance with one aspect of the present disclosure.

FIG. 7 is a process diagram of an exemplary sub-process of the gas leak detection process of FIG. 6 that may be used to determine a location of a gas leak in accordance with one aspect of the present disclosure.

FIG. 8 is a process diagram of an exemplary gas leak detection process using an artificial intelligence system in accordance with one aspect of the present invention.

### DETAILED DESCRIPTION

Before explaining at least one embodiment of the disclosure in detail, it is to be understood that the disclosure is not limited in its application to the details of construction, experiments, exemplary data, and/or the arrangement of the components set forth in the following description or illustrated in the drawings unless otherwise noted.

The systems and methods as described in the present disclosure are capable of other embodiments or of being practiced or carried out in various ways. Also, it is to be understood that the phraseology and terminology employed herein is for purposes of description, and should not be regarded as limiting.

The following detailed description refers to the accompanying drawings. The same reference numbers in different drawings may identify the same or similar elements.

As used in the description herein, the terms “comprises,” “comprising,” “includes,” “including,” “has,” “having,” or any other variations thereof, are intended to cover a non-exclusive inclusion. For example, unless otherwise noted, a process, method, article, or apparatus that comprises a list of elements is not necessarily limited to only those elements, but may also include other elements not expressly listed or inherent to such process, method, article, or apparatus.

Further, unless expressly stated to the contrary, “or” refers to an inclusive and not to an exclusive “or”. For example, a condition A or B is satisfied by one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more, and the singular also includes the plural unless it is obvious that it is meant otherwise. Further, use of the term “plurality” is meant to convey “more than one” unless expressly stated to the contrary.

As used herein, any reference to “one embodiment,” “an embodiment,” “some embodiments,” “one example,” “for example,” or “an example” means that a particular element, feature, structure or characteristic described in connection with the embodiment is included in at least one embodiment. The appearance of the phrase “in some embodiments” or “one example” in various places in the specification is not necessarily all referring to the same embodiment, for example.

Circuitry, as used herein, may be analog and/or digital components, or one or more suitably programmed processors (e.g., microprocessors) and associated hardware and software, or hardwired logic. Also, “components” may perform one or more functions. The term “component” may include hardware, such as a processor (e.g., microprocessor), a combination of hardware and software, and/or the like. Software may include one or more computer executable instructions that when executed by one or more components cause the component to perform a specified function. It should be understood that the algorithms described herein may be stored on one or more non-transitory memory. Exemplary non-transitory memory may include random access memory, read only memory, flash memory, and/or the like. Such non-transitory memory may be electrically based, optically based, and/or the like.

Gas threshold value, as used herein, refers to a concentration of gas that is calculated using a volumetric mixing ratio and expressed in parts per million (ppm).

Acoustic threshold value, as used herein, refers to a sound intensity level measured in decibels (dB) and may include a limitation to a frequency or frequency range measured in hertz (Hz). For example, in one embodiment, the acoustic threshold value may be an intensity of a measured background noise at a field site plus a value such as 6 dB. In another exemplary embodiment, the acoustic threshold value may be an intensity of at least 80 dB occurring between 20 kHz and 100 kHz.

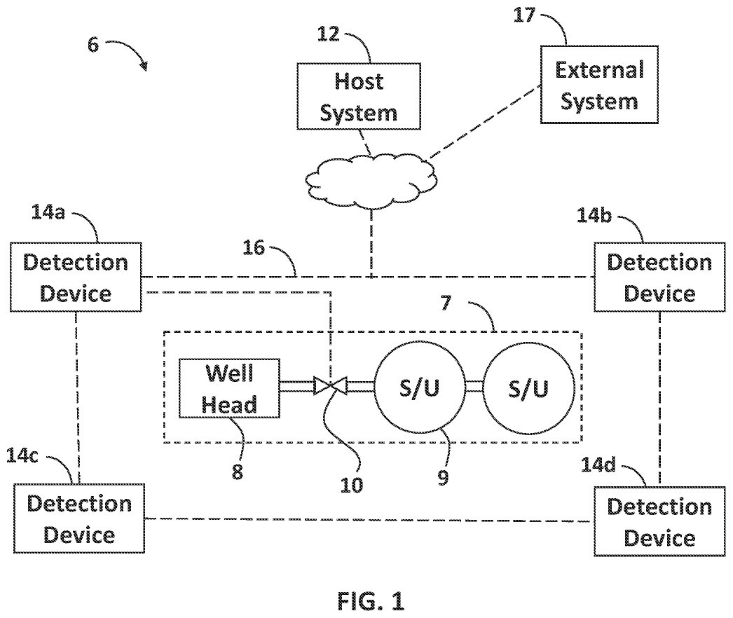

Referring now to the Figures, and in particular to FIG. 1, shown therein is a diagrammatic view of hardware forming an exemplary embodiment of a system 6 for detecting gas leaks at a field site 7 constructed in accordance with the present disclosure. Among other components, the field site 7 may have a well 8 connected to at least one storage unit 9 (only one of which is numbered in the figures) with a valve 10 situated between the well head 8 and the storage unit 9. It should be noted that the valve 10 may be any device capable of “shutting in” the well 8.

The system 6 is further provided with at least one host system 12 (hereinafter “host system 12”), a plurality of detection devices 14a-14d (hereinafter “detection device 14”), and a network 16. In some embodiments, the system 6 may include at least one external system 17 (hereinafter “external system 17”) for use by an administrator to add, delete, or modify user information, provide management reporting, manage information, or update the host system 12 and/or the detection device 14. The system 6 may be a system or systems that are able to embody and/or execute the logic of the processes described herein. Logic embodied in the form of software instructions and/or firmware may be executed on any appropriate hardware. For example, logic embodied in the form of software instructions and/or firmware may be executed on a dedicated system or systems, on a personal computer system, on a distributed processing computer system, and/or the like. In some embodiments, logic may be implemented in a stand-alone environment operating on a device and/or logic may be implemented in a networked environment such as a distributed system using multiple devices and/or processors as depicted in FIG. 1, for example.

The host system 12 of the system 6 may include a single processor or multiple processors working together or independently to perform a task. In some embodiments, the host system 12 may be partially or completely network-based or cloud based. The host system 12 may or may not be located in single physical location. Additionally, multiple host systems 12 may or may not necessarily be located in a single physical location.

In some embodiments, the system 6 may be distributed, and include at least one host system 12 communicating with one or more detection devices 14 via the network 16. As used herein, the terms “network-based,” “cloud-based,” and any variations thereof, are intended to include the provision of configurable computational resources on demand via interfacing with a computer and/or computer network, with software and/or data at least partially located on a computer and/or computer network.

The host system 12 may be capable of interfacing and/or communicating with the detection device 14 and the external system 17 via the network 16. For example, the host system 12 may be configured to interface by exchanging signals (e.g., analog, digital, optical, and/or the like) via one or more ports (e.g., physical ports or virtual ports) using a network protocol, for example. Additionally, each host system 12 may be configured to interface and/or communicate with other host systems 12 directly and/or via the network 16, such as by exchanging signals (e.g., analog, digital, optical, and/or the like) via one or more ports.

The network 16 may permit bi-directional communication of information and/or data between the host system 12, the detection device 14, and/or the external system 17. The network 16 may interface with the host system 12, the detection device 14, and/or the external system 17 in a variety of ways. For example, in some embodiments, the network 16 may interface by optical and/or electronic interfaces, and/or may use a plurality of network topographies and/or protocols including, but not limited to, Ethernet, TCP/IP, circuit switched path, Bluetooth, combinations thereof, and/or the like. For example, in some embodiments, the network 16 may be implemented as the World Wide Web (or Internet), a local area network (LAN), a wide area network (WAN), a metropolitan network, a 4G network, a 5G network, a satellite network, a radio network, an optical network, a cable network, a public switch telephone network, an Ethernet network, combinations thereof, and the like, for example. Additionally, the network 16 may use a variety of network protocols to permit bi-directional interface and/or communication of data and/or information between the host system 12, the detection device 14 and/or the external system 17.

In some embodiments, the external system 17 may optionally communicate with the host system 12. For example, in one embodiment of the system 6, the external system 17 may supply data transmissions via the network 16 to the host system 12 regarding real-time or substantially real-time events (e.g., user updates, threshold value updates, and/or sensor updates). Data transmission may be through any type of communication including, but not limited to, speech, visuals, signals, textual, and/or the like. It should be noted that the external system 17 may be the same type and construction as the host system 12 or implementations of the external system 17 may include, but are not limited to a personal computer, a cellular telephone, a smart phone, a network-capable television set, a tablet, a laptop computer, a desktop computer, a network-capable handheld device, a server, a digital video recorder, a wearable network-capable device, and/or the like.

The detection devices 14a-14d may be deployed around the field site 7 which may be an oil and/or gas site, for example. The detection devices 14a-14d may be deployed at known locations around the field site 7 and employ methods that will be described in more detail herein to quickly and autonomously determine an existence and/or location of a gas leak at the field site 7. While the system 6 is shown as having four detection devices 14a-14d, it should be noted that in some embodiments, the system 6 may have any number of detection devices 14 deployed in and around the field site 7. Furthermore, while the system 6 is shown placing the detection devices 14 around the perimeter of the field site 7, detection devices 14 may also be place in the interior such as near the components 8, 9, and/or 10.

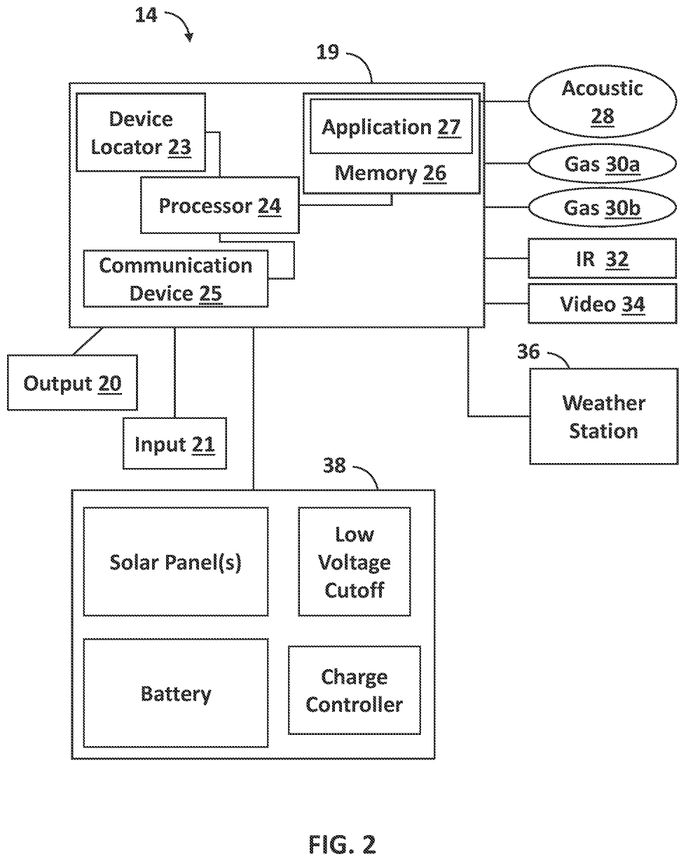

Referring now to FIG. 2, shown therein is a diagrammatic view of an exemplary embodiment of the detection device 14 which may be provided with a processing unit 19, one or more output devices 20 (hereinafter “output device 20”), one or more input devices 21 (hereinafter “input device 21”). The processing unit 19 may be provided with a device locator 23, one or more processors 24 (hereinafter “processor 24”), one or more communication devices 25 (hereinafter “communication device 25”) capable of interfacing with the network 16, one or more non-transitory computer readable memory 26 (hereinafter “memory 26”) storing processor executable code such as application 27. The detection device 14 may also include an acoustic sensor 28, at least one gas sensor 30a and 30b (hereinafter “gas sensor 30”), an infrared sensor 32 (hereinafter “IR sensor 32”), a video sensor 34, a weather station 36, and a power source 38. In some embodiments such as those installed in remote locations, for instance, the power source 38 may be provided with one or more solar panels 40 (hereinafter “solar panel 40”), one or more battery 42 (hereinafter “battery 42”), a charge controller 44, and a low voltage cutoff switch 46. Each element of the detection device 14 may be partially or completely network-based, and the elements may or may not be located in a single physical location. Any processing element, such as the processor 24 can be implemented locally or can be cloud based.

The processing unit 19 may be a single-board computer such as, for instance, a Raspberry Pi® or other similar device. The memory 26 may be implemented as a conventional non-transitory memory, such as for example, random access memory (RAM), CD-ROM, a hard drive, a solid-state drive, a flash drive, a memory card, a DVD-ROM, a disk, an optical drive, combinations thereof, and/or the like, for example.

The acoustic sensor 28, gas sensor 30, the IR sensor 32, the video sensor 34, and the weather station 36 may be connected to the processing unit 19 using connections or interfaces known in the art such as USB, General-Purpose Input/Output (GPIO), an audio jack, Bluetooth, wireless, ethernet, and the like. In some embodiments, intermediate connections, commonly referred to as “hats,” “shields,” or “expansion boards” may be used to interface the sensors with the processing unit 19.

The acoustic sensor 28 may be a sensor capable of detecting sounds in an audible spectrum (the part of the acoustic spectrum detectable by human hearing) as well as sounds in an ultrasonic spectrum (the part of the acoustic spectrum with higher frequencies than can be detected by human hearing). For instance, in some embodiments, the acoustic sensor 28 may operate in exemplary audible ranges including 5 Hz to 10 kHz, 3 Hz to 15 kHz, or 0 Hz to 30 kHz. In other embodiments, the acoustic sensor 28 may operate in exemplary ultrasonic ranges including 20 kHz to 40 kHz, 20 kHz to 60 kHz, and/or 20 kHz to 100 kHz. It should be noted that these ranges are provided for the purposes of illustration only and should not be considered limiting.

The acoustic sensor 28 may detect acoustic signals and provide the acoustic signals to the processor 24 to determine whether or not the acoustic signals are indicative of a gas leak. In some embodiments, the processor 24 may save the acoustic signals as a data file in the memory 26 for subsequent processing using the processor 24 or sent to the host system 12 for processing. The acoustic signals may be pre-processed using high/low/band-pass filtering, for instance. In some embodiments, the acoustic signals may be processed using Short-Time Fourier Transform (STFT) or Fast Fourier Transform (FFT) algorithms.

While the detection device 14 is illustrated as having one acoustic sensor 28, it should be noted that in some embodiments, the detection device 14 may include more than one acoustic sensor 28 that may be deployed in an array that allows the detection device 14 to determine a direction of acoustic signals detected by the array of acoustic sensor 28 using a time delay of when particular acoustic sensors 28 detected the acoustic signal, followed by triangulation or other methods known or developed in the art to locate the source of the acoustic signal in three-dimensional space. In these embodiments, each of the acoustic sensors 28 has a known location that can be used to determine the location of the source of the acoustic signal in three-dimensional space.

The gas sensor 30 may be a methane gas sensor that directly detects the presence of methane. The gas sensor 30 may be affected by wind direction and speed. Because wind direction and speed are variable, multiple gas sensors 30 are generally required in order to effectively determine the presence of a gas leak at a site such as field site 7. When coupled with wind data, such wind direction and speed, which may be gathered using weather station 36, a remotely located weather station, or area weather data, for instance, it is possible for the processor 24 to determine a general or possible location of a gas leak as will be described further herein.

The gas sensor 30 may be able to detect concentrations of gas in the air in a range from 1 ppm to 10,000 ppm. The gas sensor 30 may be any type of gas sensor such as an optical sensor, a calorimetric sensor, a pyroelectric sensor, a semiconducting metal oxide sensor, an electrochemical sensor, and the like capable of measuring concentrations of methane gas in the air and outputting a signal to the processing unit 19 of the detection device 14 indicative of the measured concentration of methane gas in the air. In some embodiments, the detection device 14 may be provided with more than one gas sensor 30. The more than one gas sensor 30 may be of the same type or may include more than one type of gas sensor. The type and number of gas sensor 30 may be selected based on factors such as a type of gas to be sensed, environmental factors such as humidity, temperature, and wind, and/or other factors such as presences of other types of gas.

The IR sensor 32 may be configured to detect the presence of gases such as methane in the atmosphere that may indicate undesirable conditions at an oil or gas site such as the presence of a leak or an unlit flare. The IR sensor 32 may be of a type classified as either open path detection or point detection capable of detecting the presence of gases such as hydrocarbons in the air and outputting a signal to the processing unit 19 indicative of the presence of and/or a concentration of detected gas.

The image capture device 34 may be used to surveil the field site 7 to determine a presence of possible acoustic or gas sources. The image capture device 34 may be a camera or other optical recorder capable of capturing still and/or video images of the field site 7. The images captured by the image capture device 34 may be in an infrared spectrum and/or in a spectrum of light visible to the human eye. The captured images and/or video may be stored in the memory 26 and/or may be transmitted over the network 16 to be stored in the memory 50 of the host system 12. The detection device 14 and/or the host system 12 may be programmed to process the captured images and/or video to determine the presence of possible acoustic or gas sources. For instance, when the acoustic sensor 28 detects a sound, detection device 14 may cause the image capture device 34 to capture images and/or video of the field site 7 which can be analyzed to determine if there is a possible source of the sound at the field site 7 that is not a gas leak. For instance, the presence of a vehicle or person may be a possible source of the sound. If a possible source of the sound is present, the detection device 14 may assign a lower priority and/or a higher threshold value to the sound to lower the possibility of a false alarm.

The memory 26 may store an application 27 that, when executed by the processor 24, causes the detection device 14 to automatically and without user intervention perform certain tasks such as analyzing and making decisions based on data collected from the field site 7 by the sensors of the detection device 14. The application 27 may be programmed to cause the processor 24 to analyze signals received from the sensors to determine a presence or possible presence of a gas leak and send an alert, an alarm, or other indicator via the network 16 to a recipient such as a well dispatch that indicates the presence of the gas leak as will be described further herein.

The device locator 23 may be capable of determining the position of the detection device 14. For example, implementations of the device locator 23 may include, but are not limited to, a Global Positioning System (GPS) chip, software-based device triangulation methods, network-based location methods such as cell tower triangulation or trilateration, the use of known-location wireless local area network (WLAN) access points using the practice known as “wardriving”, or a hybrid positioning system combining two or more of the technologies listed above.

The input device 21 may be capable of receiving information input from the user and/or another processor, and transmitting such information to other components of the detection device 14 and/or the network 16. The input device 21 may include, but are not limited to, implementation as a keyboard, touchscreen, mouse, trackball, microphone, fingerprint reader, infrared port, slide-out keyboard, flip-out keyboard, cell phone, PDA, remote control, fax machine, wearable communication device, network interface, combinations thereof, and/or the like, for example.

The output device 20 may be capable of outputting information in a form perceivable by the user. For example, implementations of the output device 20 may include, but are not limited to, a computer monitor, a screen, a touchscreen, a speaker, a website, a television set, a smart phone, a PDA, a cell phone, a fax machine, a printer, a laptop computer, combinations thereof, and the like, for example. It is to be understood that in some exemplary embodiments, the input device 21 and the output device 20 may be implemented as a single device, such as, for example, a touchscreen of a computer, a tablet, or a smartphone.

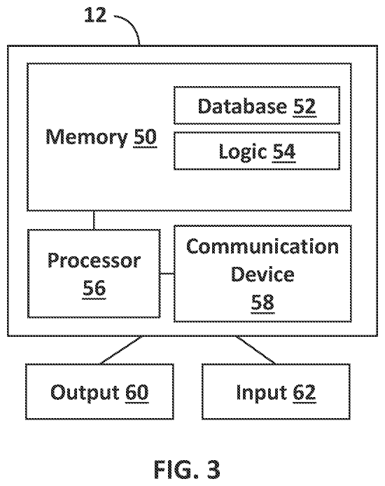

Referring now to FIG. 3, shown therein is a diagrammatic view of an exemplary embodiment of the host system 12. In the illustrated embodiment, the host system 12 is provided with non-transitory computer readable storage memory 50 (hereinafter “memory 50”) storing one or more databases 52 (hereinafter “database 52”) and program logic 54, one or more processors 56 (hereinafter “processor 56”), a communication device 58 capable of interfacing with the network 16, an input device 60, and an output device 62. The program logic 54, the database 52, and the communication device 58 are accessible by the processor 56 of the host system 12. It should be noted that as used herein, program logic 54 is another term for instructions which can be executed by the processor 24 or the processor 56.

The database 52 may be a relational database or a non-relational database. Examples of such databases comprise, DB2®, Microsoft® Access, Microsoft® SQL Server, Oracle®, mySQL, PostgreSQL, MongoDB, Apache Cassandra, and the like. It should be understood that these examples have been provided for the purposes of illustration only and should not be construed as limiting the presently disclosed inventive concepts. The database 52 can be centralized or distributed across multiple systems.

In some embodiments, the host system 12 may comprise one or more processors 56 working together, or independently to, execute processor executable code stored on the memory 50. Each element of the host system 12 may be partially or completely network-based or cloud-based, and may or may not be located in a single physical location.

The processor 56 may be implemented as a single processor or multiple processors working together, or independently, to execute the program logic 54 as described herein. It is to be understood, that in certain embodiments using more than one processor 56, the processors 35 may be located remotely from one another, located in the same location, or comprising a unitary multi-core processor. The processors 35 may be capable of reading and/or executing processor executable code and/or capable of creating, manipulating, retrieving, altering, and/or storing data structures into the memory 50.

Exemplary embodiments of the processor 56 may include, but are not limited to, a digital signal processor (DSP), a central processing unit (CPU), a field programmable gate array (FPGA), a microprocessor, a multi-core processor, combinations, thereof, and/or the like, for example. The processor 56 may be capable of communicating with the memory 50 via a path (e.g., data bus). The processor 56 may be capable of communicating with the input device 60 and/or the output device 62.

The processor 56 may be further capable of interfacing and/or communicating with the detection device 14 and/or the external system 17 via the network 16. For example, the processor 56 may be capable of communicating via the network 16 by exchanging signals (e.g., analog, digital, optical, and/or the like) via one or more ports (e.g., physical or virtual ports) using a network protocol to provide updated information to the application 27 executed on the detection device 14.

The memory 50 may be capable of storing processor executable code. Additionally, the memory 50 may be implemented as a conventional non-transitory memory, such as for example, random access memory (RAM), CD-ROM, a hard drive, a solid-state drive, a flash drive, a memory card, a DVD-ROM, a disk, an optical drive, combinations thereof, and/or the like, for example.

In some embodiments, the memory 50 may be located in the same physical location as the host system 12, and/or one or more memory 50 may be located remotely from the host system 12. For example, the memory 50 may be located remotely from the host system 12 and communicate with the processor 56 via the network 16. Additionally, when more than one memory 50 is used, a first memory 50 may be located in the same physical location as the processor 56, and additional memory 50 may be located in a location physically remote from the processor 56. Additionally, the memory 50 may be implemented as a “cloud” non-transitory computer readable storage memory (i.e., one or more memory 50 may be partially or completely based on or accessed using the network 16).

The input device 60 of the host system 12 may transmit data to the processor 56 and may be similar in construction and function as the input device 21 of the detection device 14. The input device 60 may be located in the same physical location as the processor 56, or located remotely and/or partially or completely network-based. The output device 60 of the host system 12 may transmit information from the processor 56 to a user, and may be similar in construction and function as the output device 20 of the detection device 14. The output device 60 may be located with the processor 56, or located remotely and/or partially or completely network-based.

The memory 50 may store processor executable code and/or information comprising the database 52 and program logic 54. In some embodiments, the processor executable code may be stored as a data structure, such as the database 52 and/or data table, for example, or in non-data structure format such as in a non-compiled text file.

The program logic 54 may be a program in the form of artificial intelligence which makes it possible for the program logic 54 to learn from experience and adapt to more accurately determine the presence of a gas leak based on input data from sensors. The artificial intelligence may be of a type known as a neural network capable of deep learning. Exemplary neural networks include perceptron, feed forward neural network, Multilayer Perceptron, Convolutional Neural Network, Radial Basis Functional Neural Network, Recurrent Neural Network, LSTM—Long Short-Term Memory, Sequence to Sequence Models, and Modular Neural Network, for instance. It should be noted that these examples are provided for illustrative purposes and should not be considered as limiting.

In some embodiments, the program logic 54 may be trained using datasets for acoustic pattern recognition and may be referred to as a pretrained audio neural network (PANN). The datasets may include background noises common at sites such as field site 7 where the system 6 may be deployed that allows the PANN to differentiate between background noises and sounds that may be indicative of a gas leak.

The program logic 54 may use machine learning to classify input signals from sensors such as acoustic sensor 28, gas sensor 30, IR sensor 32, video sensor 34, and/or weather station 36 and determine if the input signals are collectively indicative of a gas leak. Initially, the program logic 54 may be programmed with threshold values for each type of data input. As time goes on, the artificial intelligence may adjust these threshold values based on past experiences and/or current inputs. For instance, when the program logic 54 receives input indicative of a sound that is above an initial threshold value, the program logic 54 may use data input from another sensor such as image capture device 34 to determine if the sound may be coming from a source other than a gas leak such as, for instance, the presence of a person or vehicle, the operation of equipment, current weather conditions, etc., and adjust the threshold value based on the presence of another possible source of the sound. To adjust the threshold value, the program logic 54 may use prior instances where the other possible source of the sound was present and adjust the threshold based on the past inputs.

The system 6 may include the application 27 executed by the processor 24 of the detection device 14 that is capable of communicating with the host system 12 via the network 16. The system 6 may include a separate program, application or “app”, or a widget, each of which may correspond to instructions stored in the memory 26 of the detection device 14 for execution by the processor 24 of the detection device 14. Alternately, the system 6 may include instructions stored in the memory 50 of the host system 12 for execution by the processor 56 of the host system 12 with results sent via the network 16 to be displayed on the output device 20 of the detection device 14.

The application 27 of the detection device 14 may be programmed with threshold values for each type of signal input from sensors such as the acoustic sensor 28, the gas sensor 30, the IR sensor 32, the video sensor 34, and/or the weather station 36. The application 27 may use those threshold values to determine whether or not to further process the input signals and/or to determine that a gas leak is present. For instance, in an embodiment of the system 6 where final processing of the input signals is performed on the host system 12, the application 27 may be programmed to preprocess the input signals using minimum threshold values and only send input signals that are above the minimum threshold value to the host system 12 for final processing.

In another embodiment of the system 6, the detection device 14 may be programmed with a first threshold value and a second threshold value for each of the input signals. If the input signal is above the first threshold value but below the second threshold value, the application 27 may be programmed to cause detection device 14 to send the input signal to the host system 12 via the network 16 for further analysis and send an alert indicative of a warning of a possible gas leak to a predetermined recipient such as a well dispatch, for instance. If the input signal is above the second threshold value, the application 27 may be programmed to automatically send an alarm indicating that there is a gas leak via the network 16 to the predetermined recipient. As will be explained further in detail below, the application 27 may be programmed to automatically shut off the well 8 by closing the valve 10, for instance, if the input signal is above the second threshold value in addition to sending the alarm to the predetermined recipient.

In some embodiments, the application 27 may be programmed with the first and second threshold values for each sensor input and may be programmed to determine if a single sensor input is above the second threshold value and/or multiple sensor inputs are above the first and/or second threshold values. For instance, if an input signal from the acoustic sensor 28 is above the first threshold value but below the second threshold value and an input signal from the gas sensor 30 is above the first threshold value but below the second threshold value, the application 27 may be programmed to send an alarm indicating a gas leak to the predetermined recipient. In this case, input from a single sensor above the first threshold value but below the second threshold value alone would not have triggered the alarm. However, input signals from multiple sensors above the first threshold value but below the second threshold value will trigger the alarm. In this example, if a signal from either the acoustic sensor 28 or the gas sensor 30 was above the second threshold value, the application 27 would automatically trigger the alarm and send a signal to the predetermined recipient indicative of the alarm.

In some embodiments, the application 27 may be an artificial intelligence application and may be programmed and/or trained as described above with reference to program logic 54.

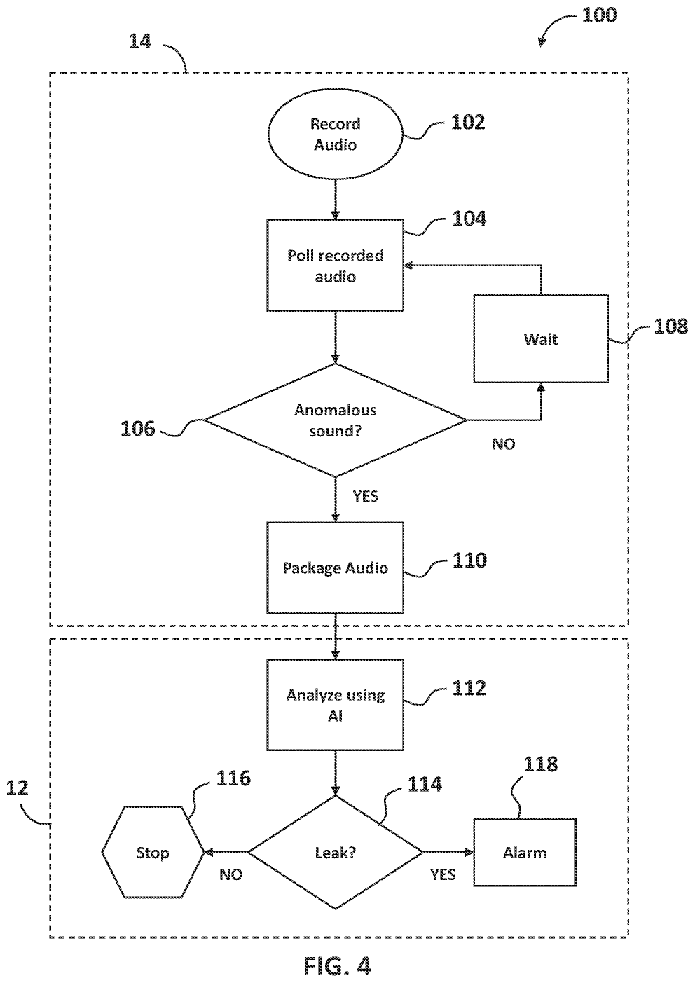

Referring now to FIG. 4, shown therein is an exemplary process diagram illustrating a process 100 of detecting a gas leak using audio signals recorded at a site such as field site 7 using the detection device 14. In step 102, the process 100 begins by recording audio (which may be in an audible spectrum, in an ultrasonic spectrum, or both).

At predetermined intervals, the application 27 causes the detection device 14 to poll the recorded audio and pass at least a portion of the recorded audio through a filter in step 104. For instance, the recorded audio may be filtered using a low-pass filter, a high-pass filter, a band-pass filter, a notch filter, or the like.

In decision step 106, the application 27 causes the processing unit 19 of the detection device 14 to analyze the filtered audio to determine if there is an anomalous sound in the recorded audio.

If no anomalous sound is detected, in step 108 the detection device 14 waits a predetermined amount of time before returning to step 104 and passing a new set of recorded audio through the filter in step 104.

If an anomalous sound is detected, in step 110 the detection device 14 packages and sends at least a portion of the filtered audio to the host system 12 over the network 16.

In step 112, the host system 12 analyzes the filtered audio to determine if a gas leak is present. The host system 12 may use artificial intelligence embodied in program logic 54 as described above to analyze the filtered audio.

In decision step 114, the host system 12 determines if a leak has been detected. If a leak is not detected, in step 116 the process ends on the host system 12. In some embodiments, the host system 12 may be programmed to send a signal to the detection device 14 indicating the end of the process which may cause the detection device 14 to resume the process 100 at step 104.

If the host system 12 determines that a leak has been detected in step 114, in step 118 the host system 12 is programmed to cause an alarm to be sent to at least one predetermined recipient or a signal to shut off the well 8 by closing the valve 10. The alarm can be indicative of a gas leak.

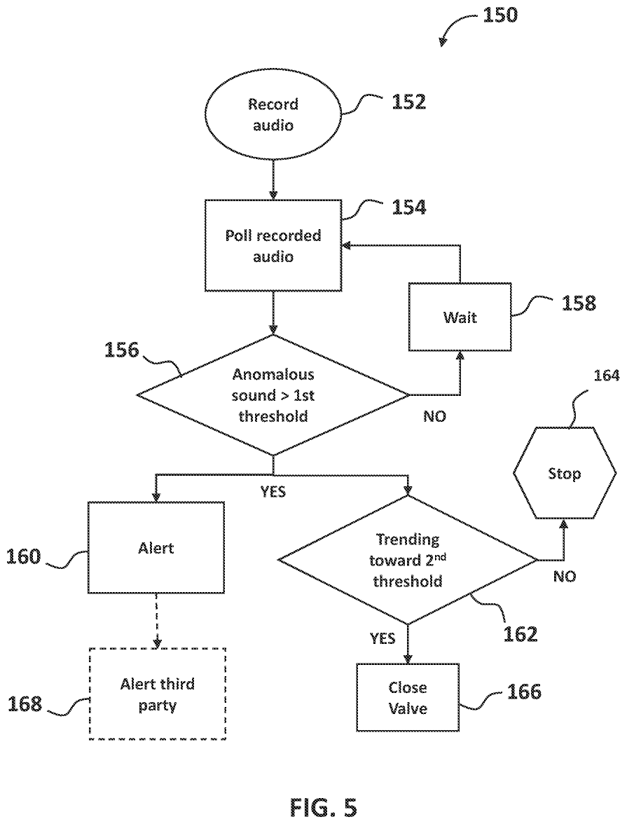

Referring now to FIG. 5, shown therein is an exemplary process diagram illustrating a process 150 of detecting a gas leak using audio recorded at a site such as field site 7 using the detection device 14. In step 152, the process 150 begins by recording audio (which may be in an audible spectrum, in an ultrasonic spectrum, or both).

At repeated predetermined intervals of time, the detection device 14 polls the recorded audio and passes at least a portion of the recorded audio through a filter in step 154.

In decision step 156, the application 27 causes the processing unit 19 of the detection device 14 to analyze the filtered audio to determine if there is a gas leak by determining if an anomalous sound that is above a first threshold value is present at more than one predetermined interval of time.

If no anomalous sound is detected at more than one predetermined interval of time, in step 158 the detection device 14 waits a predetermined amount of time before returning to step 154.

If an anomalous sound above the first threshold value is detected at more than one predetermined interval of time, in step 160 the detection device 14 sends an alert to a predetermined recipient over the network 16. The predetermined recipient may be a well dispatch, for instance. In optional step 168, the well dispatch and/or the detection device 14 may be programmed to send an alert to a third party such as a repair person

In some embodiments, if an anomalous sound above the first threshold value is detected at one or more intervals, in decision step 162 the detection device 14 may further be programmed to determine if the anomalous sound is trending toward and/or is above a second threshold value.

If the detection device 14 determines that the anomalous sound is not trending toward and/or is not above the second threshold value, in step 164 the process 150 ends.

If the detection device 14 determines that the anomalous sound is trending toward and/or is above the second threshold value, in step 166 the detection device 14 may be programmed to automatically close the valve 10 or other device to prevent further gas from leaking from the well 8, for instance.

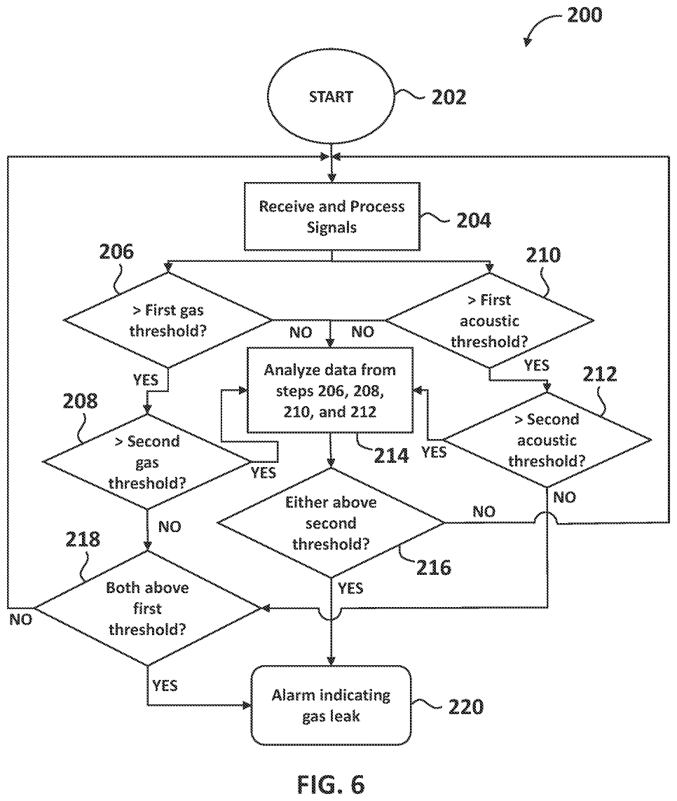

Referring now to FIG. 6, shown therein is a process 200 for detecting a gas leak using data from both the acoustic sensor 28 and the gas sensor 30 of the detection device 14. In step 202, the processing unit 19 is programmed to start the process 200.

In step 204, the processing unit 19 receives and processes input signals from the acoustic sensor 28 and the gas sensor 30 using one or more of the processes described above.

In decision step 206, the processing unit 19 analyzes the processed input signal from the gas sensor 30 to determine if the processed input signal is above a first gas threshold value. If the processed input signal is above the first gas threshold, the processing unit 19 proceeds to step 208. If the processed input signal is below the first gas threshold, the processing unit 19 saves data indicative of the processed input signal being below the first gas threshold for further processing in step 214.

In step 208, the processing unit 19 analyzes the processed input signal from the gas sensor 30 to determine if the processed input signal is above a second gas threshold value. If the processed input signal is above the second gas threshold, the processing unit 19 saves data indicative of the processed input signal being above the second gas threshold for further processing in step 214. If the processed input signal is below the second gas threshold, the processing unit 19 saves data indicative of the processed input signal being below the second gas threshold for further processing in step 218.

In decision step 210, the processing unit 19 analyzes the processed input signal from the acoustic sensor 28 to determine if the processed input signal is above a first acoustic threshold value. If the processed input signal is above the first acoustic threshold, the processing unit 19 proceeds to step 212. If the processed input signal is below the first acoustic threshold, the processing unit 19 saves data indicative of the processed input signal being below the first acoustic threshold for further processing in step 214.

In decision step 212, the processing unit 19 analyzes the processed input signal from the acoustic sensor 28 to determine if the processed input signal is above a second acoustic threshold value. If the processed input signal is above the second acoustic threshold, the processing unit 19 saves data indicative of the processed input signal being above the second acoustic threshold for further processing in step 214. If the processed input signal is below the second acoustic threshold, the processing unit 19 saves data indicative of the processed input signal being below the second acoustic threshold for further processing in step 218.

In step 214, the processing unit 19 receives and analyzes data from steps 206, 208, 210, and 212. In decision step 216, if the processing unit 19 determines that both the processed input signal from the acoustic sensor 28 is below the first acoustic threshold value and the processed input signal from the gas sensor 30 is below the first gas threshold value, the processing unit 19 is programmed to begin the process 200 over. In decision step 216, if the processing unit 19 determines that either the processed input signal from the acoustic sensor 28 is above the second acoustic threshold value or the processed input signal from the gas sensor 30 is above the second gas threshold value, the processing unit 19 is programmed to cause an alarm to be sent to at least one predetermined recipient or a signal to shut off the well 8 by closing the valve 10 in step 220. The alarm can be indicative of a gas leak.

In decision step 218, the processing unit 19 determines if it has received data from both steps 208 and 212 indicating that the processed input signal from the gas sensor 30 is above the first gas threshold value and the processed input signal from the acoustic sensor 28 is above the first acoustic threshold value. If the processing unit 19 has received data indicating that both the processed input signal from the gas sensor 30 is above the first gas threshold value and the processed input signal from the acoustic sensor 28 is above the first acoustic threshold value, in step 218 the processing unit 19 causes the alarm to be sent to at least one predetermined recipient in step 220, the alarm indicative of a gas leak. If the processing unit 19 has not received data from both steps 208 and 212, the processing unit 19 determines that at least one of the processed input signal from the gas sensor 30 is below the first gas threshold value and/or the processed input signal from the acoustic sensor 28 is below the first acoustic threshold value and the processing unit 19 causes the process 200 to start over.

The steps of the process 200 have been numbered for the sake of illustration and the numerical order in which they are described is not necessarily the order in which they are performed. For instance, steps 206 and 210 may be performed in parallel.

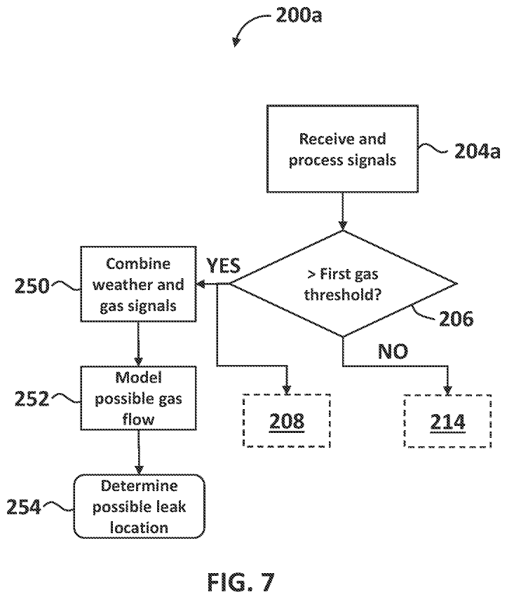

Referring now to FIG. 7, shown therein is an exemplary sub-process 200a of process 200 that may be used to determine a probable location of a gas leak. The sub-process 200a is similar to the process 200 described above. Therefore, in the interest of brevity only the differences will be described in detail herein.

In step 204a, the processing unit 19 receives and processes input signals from the acoustic sensor 28, the gas sensor 30, and the weather station 36 using one or more of the processes described above.

In decision step 206, the processing unit 19 analyzes the processed input signal from the gas sensor 30 to determine if the processed input signal is above a first gas threshold value. If the processed input signal is above the first gas threshold, the processing unit proceeds to steps 208 and 250. If the processed input signal is below the first gas threshold, the processing unit 19 saves data indicative of the processed input signal being below the first gas threshold for further processing in step 214.

In step 250, the processing unit 19 causes the processed input signals from the gas sensor 30 and the weather station 36 to be combined. It should be noted that the processed input signals from the weather station 36 may include local weather data, wind speed, wind direction, and other atmospheric conditions such as solar intensity, temperature, and relative humidity.

In step 252, the processing unit 19 may further process the combined data from the gas sensor 30 and the weather station 36 to produce a model of possible gas flow patterns based on the inputs received from the gas sensor 30 and the weather station 36. The processing unit 19 may use an inverse atmospheric dispersion model and/or machine learning model to process the combined data. Furthermore, the processing unit 19 may combine signals from multiple gas, acoustic, or other sensors such as sensors 14a-14d to process the combined data.

As used herein, inverse atmospheric dispersion model means a model that uses physical dispersion of a gas in the atmosphere and then back-calculates to a probable source location based on the atmospheric conditions and the signal at a known location.

As used herein, machine learning model refers to a model (e.g., linear regression or a neural net), which has been trained using training datasets or known data.

In step 254, the processing unit 19 determines a probable leak location. The probable leak location may be transmitted to a predetermined recipient. It should be noted the probable leak location may be transmitted alone, in addition to, or in lieu of the alarm sent in step 220 described above. Further, if the wellsite 7 has more than one well 8, the information regarding the probable leak location can be used to send a signal to a particular valve 10 of a series of valves 10 to close off a particular well 8.

It should be noted that while the processes 200 and 200a have been described as being performed by the detection device 14, in some embodiments one or more steps may be performed by the host system 12.

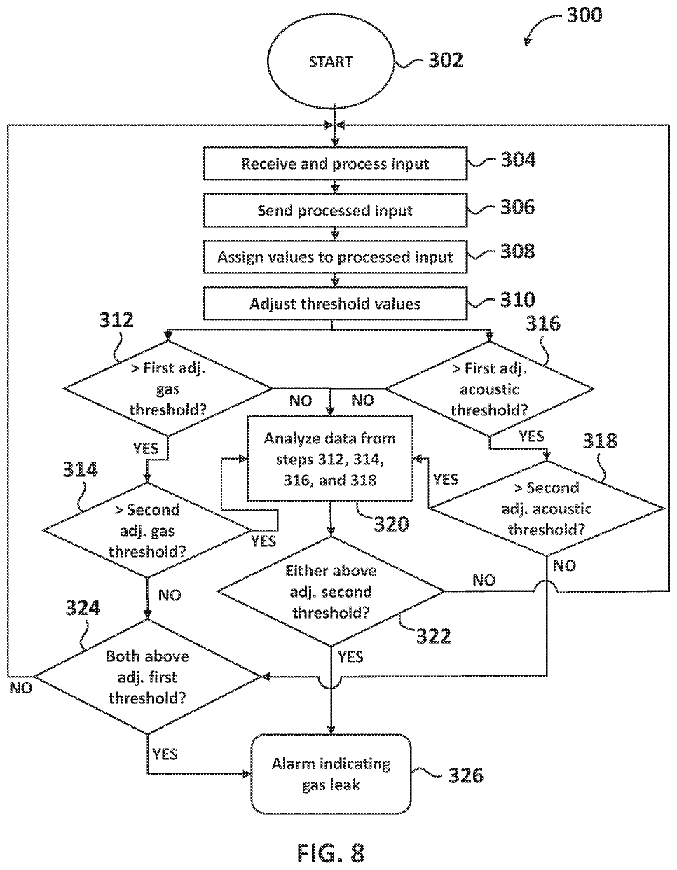

Referring now to FIG. 8, shown therein is a process 300 for gas leak detection which may be performed by the system 6. In step 302, the process 300 begins on the processing unit 19. In step 304, the processing unit 19 receives and processes input from the acoustic sensor 28, the gas sensor 30, the IR sensor 32, and the image capture device 34.

In step 306, the processing unit 19 sends the processed input from the acoustic sensor 28, the gas sensor 30, the IR sensor 32, and the image capture device 34 to the host system 12 over the network 16.

In step 308, the host system 12 analyzes and further processes the input from the acoustic sensor 28, the gas sensor 30, the IR sensor 32, the image capture device 34, and the weather station 36. The analysis and further processing including assigning an acoustic value to the input from the acoustic sensor 28, a gas value to the input received from the gas sensor 30, and analyzing the input from the IR sensor 32, the input from the image capture device 34, and the input from the weather station 36 to determine a presence of a possible source of the input from the acoustic sensor 28 and/or the input from the gas sensor 30. For instance, the host system 12 may be programmed to analyze the input from the image capture device 34 to determine the presence of a person, a vehicle, machinery, and/or animals at the field site 7, for instance, which may be a source of sound or gas emissions that may have been captured by the acoustic sensor 28 and/or the gas sensor 30, respectively. The host system 12 may also be programmed to analyze the input from the weather station 36 to determine possible sources of sound or gas. For instance, if the field site 7 is situated near livestock, a known source of methane emissions, that are located in a known direction relative to the field site 7, the host system 12 may be programmed to analyze a wind speed and/or direction to determine if methane emissions from the livestock could be a source of gas sensed by the gas sensor 30. In some embodiments, the analysis and further processing may be performed automatically using a machine learning model running on the host system 12, the machine learning model developed using one or more training datasets supplied to the machine learning model as well as data previously analyzed during operation of the host system 12.

In step 310, the machine learning model of the host system 12 may use the analyzed input from the IR sensor 32, the analyzed input from the image capture device 34, and/or the analyzed input from the weather station 36 to automatically adjust a first gas threshold value, a second gas threshold value, a first acoustic threshold value, and/or a second acoustic threshold value based on a determination that there was a possible source of the input received from the acoustic sensor 28 and/or the gas sensor 30 other than a gas leak. For instance, if the presence of a person or persons was detected at the field site 7 and determined to be a possible source of sound not associated with a gas leak, the first and/or the second acoustic threshold values may be increased to reduce the risk of a false alarm based on the input received from the acoustic sensor 28. In another example, if the host system 12 determines that a wind direction at the time the input was received from the gas sensor 30 would carry methane emissions from livestock located at a known location relative to the field site 7 to the gas sensor 30, the host system 12 may be programmed to increase the first and/or second gas threshold values to compensate for the possible methane blown in on the wind. These examples have been provided for the purposes of illustration only and should not be considered as limiting. The machine learning model of the host system 12 may be programmed and/or trained to identify any number of possible sources of sound and/or gas emissions that may affect the process 300 and automatically adjust the first gas threshold value, the second gas threshold value, the first acoustic threshold value, and/or the second acoustic threshold value accordingly.

In decision step 312, the host system 12 compares the gas value to the adjusted first gas threshold value to determine if the gas value is above the adjusted first gas threshold value. If the gas value is above the adjusted first gas threshold value, the host system 12 proceeds to step 314. If the gas value is below the adjusted first gas threshold, the host system 12 saves data indicative of the gas value being below the adjusted first gas threshold for further processing in step 320.

In step 314, the host system 12 analyzes the gas value to determine if the gas value is above an adjusted second gas threshold value. If the gas value is above the adjusted second gas threshold value, the host system 12 saves data indicative of the gas value being above the adjusted second gas threshold for further processing in step 320. If the gas value is below the adjusted second gas threshold, the host system 12 saves data indicative of the processed input signal being below the second gas threshold for further processing in step 324.

In decision step 316, the host system 12 analyzes the acoustic value to determine if the acoustic value is above an adjusted first acoustic threshold value. If the acoustic value is above the adjusted first acoustic threshold value, the host system 12 proceeds to step 318. If the acoustic value is below the adjusted first acoustic threshold value, the host system 12 is programmed to save data indicative of the acoustic value being below the adjusted first acoustic threshold value for further processing in step 320.

In decision step 318, the host system 12 analyzes the acoustic value to determine if the acoustic value is above an adjusted second acoustic threshold value. If the acoustic value is above the adjusted second acoustic threshold value, the host system 12 saves data indicative of the acoustic value being above the adjusted second acoustic threshold value for further processing in step 320. If the acoustic value is below the adjusted second acoustic threshold value, the host system 12 saves data indicative of the acoustic value being below the adjusted second acoustic threshold value for further processing in step 324.

In step 320, the host system 12 receives and analyzes data from steps 312, 314, 316, and 318. In decision step 322, if the host system 12 determines that both the acoustic value is below the adjusted first acoustic threshold value and the gas value is below the adjusted first gas threshold value, the host system 12 is programmed to begin the process 300 over. In decision step 322, if the host system 12 determines that either the acoustic value is above the adjusted second acoustic threshold value or the gas value is above the adjusted second gas threshold value, the host system 12 is programmed to cause an alarm to be sent to at least one predetermined recipient or a particular valve 10 to shut off at least one well 8 in step 326. The alarm is indicative of a gas leak.

In decision step 324, the host system 12 determines if it has received data from both steps 314 and 318 indicating that the gas value is above the adjusted first gas threshold value but below the adjusted second gas threshold value and the acoustic value is above the adjusted first acoustic threshold value but below the adjusted second acoustic threshold value. If the host system 12 has received data indicating that both the gas value is above the adjusted first gas threshold value and the acoustic value is above the adjusted first acoustic threshold value in step 324, the host system 12 causes the alarm to be sent to at least one predetermined recipient or a particular valve 10 to shut off at least one well 8 in step 326. If the host system 12 has not received data from both steps 314 and 318, the host system 12 determines that at least one of the gas values is below the adjusted first gas threshold value and/or the acoustic value is below the adjusted first acoustic threshold value and the host system 12 causes the process 300 to start over.

From the above description, it is clear that the inventive concept(s) disclosed herein are well adapted to carry out the objects and to attain the advantages mentioned herein, as well as those inherent in the inventive concept(s) disclosed herein. While the embodiments of the inventive concept(s) disclosed herein have been described for purposes of this disclosure, it will be understood that numerous changes may be made and readily suggested to those skilled in the art which are accomplished within the scope and spirit of the inventive concept(s) disclosed herein.

1. A system, comprising:
at least one gas sensor;
an acoustic sensor;
a processing unit having a processor and a non-transitory computer readable storage media storing instructions indicative of a process for detecting a gas leak requiring data from both the acoustic sensor and the at least one gas sensor, a first gas threshold value, and a first acoustic threshold value, the processing unit configured to receive signals from at least one gas sensor above and below the first gas threshold value, the processing unit also configured to receive signals from the acoustic sensor above and below the first acoustic threshold value; and
wherein the instructions indicative of the process, when executed, cause the processor of the processing unit to analyze a first signal received from at least one gas sensor and a second signal received from the acoustic sensor to determine a presence of a gas leak, the presence of the gas leak determined if both the first signal received from the at least one gas sensor is above the first gas threshold value and the second signal received from the acoustic sensor is above the first acoustic threshold value.

2. The system of claim 1, further comprising a communication device and wherein the instructions further cause the processing unit to send an alarm to a predetermined recipient when the processing unit determines the presence of the gas leak.

3. The system of claim 2, wherein the instructions further cause the processing unit to send a signal via the communication device to a valve of a well causing the valve to close when the processing unit determines the presence of the gas leak.

4. A system, comprising:
at least one gas sensor;
an acoustic sensor;
a processing unit having a processor and a non-transitory computer readable storage media storing instructions indicative of a process for detecting a gas leak using data from both the acoustic sensor and the at least one gas sensor, the non-transitory computer readable storage media also storing a first gas threshold value, a second gas threshold value, a first acoustic threshold value and a second acoustic threshold value, the second gas threshold value higher than the first gas threshold value, the second acoustic threshold value higher than the first acoustic threshold value, the processing unit configured to receive signals from at least one gas sensor above and below the first gas threshold value and the second gas threshold value, the processing unit also configured to receive signals from the acoustic sensor above and below the first acoustic threshold value and the second acoustic threshold value;
wherein the instructions indicative of the process, when executed, cause the processor of the processing unit to analyze the signals received from at least one gas sensor and the acoustic sensor to determine the presence of a gas leak, the presence of the gas leak determined if both at least one signal received from the at least one gas sensor is above the first gas threshold value and at least one signal received from the acoustic sensor is above the first acoustic threshold value, or the at least one signal received from the at least one gas sensor is above the second gas threshold value higher than the first gas threshold value, or at least one signal received from the acoustic sensor is above the second acoustic threshold value higher than the first acoustic threshold value.

5. The system of claim 4, further comprising a communication device and wherein the instructions further cause the processing unit to send an alarm to a predetermined recipient when the processing unit determines the presence of the gas leak.

6. The system of claim 1, wherein a location of each of at least one gas sensors is stored in the non-transitory computer readable storage media, the system further comprising:
a weather station configured to capture atmospheric conditions at a field site; and
wherein the instructions further cause the processing unit to analyze signals received from the weather station, the signals indicative of the atmospheric conditions at the field site and determine a probable location of the gas leak using the atmospheric conditions and the location of the at least one gas sensor.

7. The system of claim 1, further comprising an image capture device configured to capture an image at the field site.

8. The system of claim 7, wherein the image capture device captures images in an infrared spectrum.

9. The system of claim 7, wherein the image capture device captures images in a spectrum visible to the human eye.

10. The system of claim 7, wherein the image capture device captures video images.

11. A method of identifying a gas leak, comprising:
receiving input from at least one gas sensor, the input indicative of a presence of an amount of gas in the air at a field site;
analyzing the input from at least one gas sensor to determine if the amount of gas in the air is above a first gas threshold value stored in a non-transitory computer readable medium;
receiving input from an acoustic sensor, the input indicative of a sound at the field site;
analyzing the input from the acoustic sensor to determine if the sound is above a first acoustic threshold value stored in a non-transitory computer readable medium; and
sending an alarm to a predetermined recipient if it is determined that both the amount of gas is above the first gas threshold value and the sound is above the first acoustic threshold value, the alarm indicative of a presence of a gas leak at the field site.

12. The method of claim 11, further comprising:
analyzing the input from the at least one gas sensor to determine if the amount of gas in the air is above a second gas threshold value stored in the non-transitory computer readable medium, the second gas threshold value higher than the first gas threshold value;
analyzing the input from the acoustic sensor to determine if the sound is above a second acoustic threshold value stored in the non-transitory computer readable medium, the second acoustic threshold value higher than the first acoustic threshold; and
sending the alarm to the predetermined recipient if it is determined that the gas is above the second gas threshold value or the sound is above the second acoustic threshold value, the alarm indicative of a presence of a gas leak at the field site.

13. The method of claim 12, wherein the first gas threshold value, the second gas threshold value, the first acoustic threshold value, and the second acoustic threshold value are predetermined.

14. The method of claim 12, wherein the first gas threshold value, the second gas threshold value, the first acoustic threshold value, and the second acoustic threshold value are automatically determined by a computer system running a machine learning model.

15. The method of claim 11, further comprising closing a valve automatically responsive to a determination that the gas is above the first gas threshold value and the sound is above the first acoustic threshold value.

16. The method of claim 12, further comprising closing a valve automatically if it is determined that the gas is above the second gas threshold value or the sound is above the second acoustic threshold value.

17. The method of claim 11, wherein a location of each of at least one gas sensors are known, the method further comprising:
receiving input from a weather station, the input indicative of atmospheric conditions at the field site; and
determining a probable location of a gas leak using the atmospheric conditions and the location of the at least one gas sensor.

18. The method of claim 11, further comprising:
receiving input from a video capture device, the input including images of the field site;
analyzing the input from the video capture device to determine the presence of a source of sound at the field site that is not a gas leak; and
not sending the alarm to the predetermined recipient if it is determined that the source of sound at the field site is not a gas leak.

19. The method of claim 18, wherein the image capture device captures images in an infrared spectrum.

20. The method of claim 18, wherein the image capture device captures images in a spectrum visible to the human eye.

21. The system of claim 1, wherein the non-transitory computer readable storage media stores the first gas threshold value, a second gas threshold value, the first acoustic threshold value, and a second acoustic threshold value, and wherein the presence of the gas leak is determined if both at least one signal received from the at least one gas sensor is between the first gas threshold value and the second gas threshold value and at least one signal received from the acoustic sensor is between the first acoustic threshold value and the second acoustic threshold value.
